(19) United States
(12) Patent Application Publication
(10) Pub. No.: US 2009/0270757 A1
(43) Pub. Date: Oct. 29, 2009

(54) THERMAL PROBE FOR QUANTITATIVE SENSORY PAIN TESTING

(76) Inventor: Miroslav Backonja, Madison, WI (US)

Correspondence Address: WARF/BHGL, P.O. Box 10395, Chicago, IL, 60610, US

(21) Appl. No.: 12/369,072

(22) Filed: Feb. 11, 2009

Related U.S. Application Data
(60) Provisional application No. 61/027,711, filed on Feb. 11, 2008.

(51) Int. Cl. A61B 5/00

(52) U.S. Cl. 600/555

(57) ABSTRACT

Devices, systems, and methods for sensory quantitative pain testing in subjects are provided. The devices, systems, and methods are particularly useful for conducting sensory tests following thermal stimuli applied to subjects in clinical settings. The devices, systems, and methods may be used for diagnosing painful neuropathies in subjects.

### CROSS-REFERENCE TO RELATED APPLICATIONS

This invention claims priority to U.S. Provisional Patent Application Ser. No. 61/027,711, filed Feb. 11, 2008, which is herein incorporated by reference.

### FIELD OF THE INVENTION

The present invention relates generally to devices, systems, and methods for sensory pain testing in subjects.

### BACKGROUND

Neuropathic pain is characterized by pain in an area of abnormal somatic sensory functions; consequently, the necessary step in making the diagnosis of neuropathic pain is assessment of neuropathic pain symptoms and the sensory examination. Patients with neuropathic pain suffer from a variety of symptoms that range from positive sensory phenomena, including spontaneous pain and various types of increased sensitivity to innocuous and noxious stimuli (known as allodynia and hyperalgesia, respectively), to negative sensory phenomena such as loss of sensation in the same affected area. It is puzzling for patients and perceived as paradox by clinicians that a patient would experience positive sensory phenomena in the same body part where the patient has a loss of sensation; however, it is the existence of both negative and positive sensory phenomena that define neuropathic pain (Backonja, 2003, Anesthesia & Analgesia 97: 785-790). In the clinical setting, sensory phenomena manifest in two ways, first spontaneously (reported by patients as symptoms), and second as elicited or evoked during application of specific stimuli (known as signs). Positive evoked phenomena, such as allodynia and hyperalgesia, have been the primary focus of preclinical investigation and it is through this laboratory research that neuropathic pain mechanisms have been elucidated over the past two decades.

In general terms it can be stated that the application of a specific stimulus leads to activation of a specific sensory channel and a pathway. The case of neuropathic pain is more complex. Science advances have led to an understanding that sensory signs such as allodynia are the result of activation of a specific mechanism, such as peripheral sensitization of high threshold mechanoreceptors. Since the pain system has so many sensory channels and pathways, to have a complete understanding of neuropathic pain one needs to study all sensory channels and pathways by applying comprehensive approaches.

Quantitative sensory testing (QST) is becoming a recognized tool for diagnosing peripheral nervous system disorders, including chronic pain and pain related to various diseases, such as diabetes and Complex Regional Pain Syndrome (CRPS). QST essentially determines the sensation and pain thresholds for cold and warm temperatures, and also the vibration sensation threshold, by stimulating the skin and comparing the results to normative values built in the software. When the applied stimulus activates stimuli-specific receptors, the nerve fibers that innervate the receptors communicate the message to the central nervous system, where feeling occurs. Several methods may be used to alter skin temperature for psychophysical testing. These include application of hot or cold liquids to a skin surface, immersion of a limb in a liquid, exposure of skin to an intense focused light or laser beam, or contacting the skin with a water circulating thermode, an ohmic heating element, or a Peltier device.

QST is also beginning to find utility in preclinical and clinical drug development. QST in combination with electrophysiology of evoked potentials, and more recently neuroimaging, has dramatically expanded our understanding of the physiology of the somatic sensory system and, more specifically, human pain physiology. Clinical applications of QST have lagged behind dramatic developments in laboratory research. There are several reasons for this, including the lack of standards for testing, the lack of normative data, and the lack of consensus and guidelines on how to interpret data from QST.

Recent efforts have attempted to overcome these obstacles. With advances in our understanding of the neuroscience of neuropathic pain, there is an opportunity to implement concepts of mechanism-based diagnosis and treatment of pain; however, truly quantitative methods for measurement are necessary before this translation from bench to bedside could be implemented. Currently available commercial devices for thermal quantitative testing are CASE IV (WR Medical Electronics, Stillwater, Minn.), MSA Thermotest (Somedic, Stockholm, Sweden) and TSA II (Medoc, Ramat Yishai, Israel); these devices are focused on the detection of pain thresholds for stimuli applied to the patient.

Like every newly developing area of clinical research, the study of neuropathic pain has an opportunity to make great progress and the application of quantitative measurements is an essential step in that direction. QST has evolved to the point of becoming a tool for both clinical research and practice. However, to date, existing devices for monitoring QST have not made the transition to point of care medicine to assist in routine therapy administration. For the most part, these devices are cumbersome, expensive, insufficiently quantitative or relatively insensitive to subtle temperature gradients. Despite a growing interest in neuropathic pain, neurologists and pain specialists do not have a standard, validated, office examination for evaluation of neuropathic pain signs to complement the neurologic, musculoskeletal, and general physical examinations. An office neuropathic pain examination focused on quantifying sensory features of neuropathic pain, ranging from deficits to allodynia and hyperalgesia, and evoked by a physiologically representative array of stimuli, will be an essential tool to monitor treatment effectiveness and for clinical investigation into the mechanisms and management of neuropathic pain. Such an examination should preferably include mapping of areas of stimulus-evoked neuropathic pain as well as standardized, reproducible QST of tactile, punctuate, pressure, and thermal modalities. Thus, it would be beneficial to provide novel devices and methods for sensory pain testing in a variety of subjects. The present invention addresses these and related needs.

### BRIEF SUMMARY

Provided are neuropathy diagnostic systems configured for use with human subjects, which include: a probe comprising a heating element; a control unit coupled with the probe; an input device operatively coupled with the control unit to enable input of a target temperature to the control unit; and a display coupled with the control unit to display the temperature of the probe; where the control unit controls the energy provided to the probe to thereby change the temperature of the probe from an initial temperature to the target temperature; and a feedback data point recorder to record the subject's indication of the intensity of heat sensation and the temperature of the probe at that point in time. In the systems, the control unit may include a comparator with which to calculate a difference between the target temperature and the effective probe temperature.

In preferred embodiments of the neuropathy diagnostic systems, the control unit calculates a difference between the target temperature and the effective probe temperature, and with that difference adjusts the effective probe temperature to substantially correspond to the target temperature. The control unit may include a single-crystal microprocessor controller. The control unit may include a thermocouple to measure the temperature. The heating element may include a high power metal film resistor capable of heating the probe to temperatures between about 30° C. and about 50° C. for a period of between about 1 second and about 10 seconds. The display may display a time period during which the probe has been applied to skin of the subject. The control unit may include a safety mechanism coupled with an analog-to-digital converter, where if the heating element malfunctions, the safety mechanism causes the display to stop displaying the effective probe temperature. The probe may include a brass cap configured to safely contact the skin of the subject.

Provided are methods for diagnosing neuropathy in subjects. The methods include: contacting the subject with a probe configured to apply heat at a range of temperatures, the probe having an initial temperature; setting a target temperature to which the probe temperature is to be changed; providing a readout of the temperature of the probe as it is changed to the target temperature; recording a plurality of feedback data points as the temperature of the probe is changed from the initial temperature to the target temperature, each feedback data point comprising the subject's indication of the intensity of heat sensation and the temperature of the probe at that point in time; and comparing the plurality of feedback data points to predetermined values, to thereby detect abnormal heat sensations, if any, by the subjects. In the practice of the methods, the plurality of the data points may be gathered over a range of temperature points of between about 30° C. and about 50° C.

The practice of the methods may include contacting the thermal probe to at least another point of the subject's skin to verify correlation with a neuropathic condition. In the practice of the methods an area of between about 1 cm² and about 10 cm² of the subject's skin may be heated using the thermal probe with a target temperature of between about 30° C. and about 50° C. In the practice of the methods, the target temperature may be applied for a period of between about 1 second and about 10 seconds. The practice of the methods may further include observing a timer of the display that displays a period of time during which the thermal probe has contacted the subject's skin. The practice of the methods may include removing the thermal probe from contact with the subject's skin after a set period of time. The practice of the methods may include the steps of contacting different points on the subject's skin, to provide a sensory map.

### BRIEF DESCRIPTION OF THE DRAWINGS

FIG. 1 is simplified block diagram of one embodiment of a thermal probe of the present invention.

FIG. 2 is a simplified flow diagram of one example of a microcode program used in the practice of the present invention.

FIG. 3 is a schematic diagram illustrating the validation of positive sensory signs by sensory symptoms.

FIG. 4 is an image of one embodiment of the device of the present invention.

### DETAILED DESCRIPTION OF THE PRESENTLY PREFERRED EMBODIMENTS

In one aspect, the present invention relates to a new device useful for conducting thermal quantitative sensory testing (QST) and also useful for testing and diagnostic protocols for the study of neuropathic pain mechanisms in a variety of subjects, including human patients. Thus, in one embodiment, the present invention provides a painful neuropathy diagnostic device. In particular, the present device can precisely deliver, for a desired period of time, any of a series of temperatures in the form of thermal stimuli within a useful clinical range. The device is designed to be portable, accurate, and includes fail-safe mechanisms to protect the tested subjects.

In another aspect, the present invention relates to a new system useful for conducting thermal QST and also useful for testing and diagnostic protocols for the study of neuropathic pain mechanisms in a variety of subjects, including human patients. The system may include the device of the present invention.

“Quantitative sensory testing (QST)”, as used herein, refers to the physiological methods of detection and quantification of physical properties of various stimuli, for example thermal stimuli. As a psychophysical test, QST requires active participation of the subject. The basic premise of QST is that physical stimuli applied to the body under normal physiological circumstances activate specific sets of receptors and generate physiological signals in specific anatomic components of the sensory nervous system. Such components include peripheral nerve fibers of specific size and conduction velocities as well as central pathways which lead to perception and to the report by the subject about the physical properties of the stimulus. Traditionally, QST is used for detection of deficits in patients with distal polyneuropathies. Most quantitative stimulation protocols have been developed for cutaneous applications, and consequently QST is frequently equated with cutaneous stimulation-related physiology. There are also quantitative stimulation procedures for deep tissues such as muscles and periostium using, for example, pressure algometers. QST can also be devised for visceral organs, using pressure manometry as the primary method of stimulation. In addition to the more commonly utilized mechanical and thermal stimuli, electrical stimulation is utilized for the same purposes of assessing the status of the somatic sensory system as well as related neuroplasticity. A review of QST in measurement of neuropathic pain phenomena and other sensory abnormalities, useful in the practice of the present invention, can be found in Backonja et al. 2009, Clinical Journal of Pain, in press. A review of non-automated quantitative methods for examination of the patient with neuropathic pain, useful in the practice of the present invention, can be found in Walk et al., 2009, Clinical Journal of Pain, in press.

QST has several basic elements common to all psychophysical methods including: 1) a subject who receives the stimulus and reports the perceived sensation produced by the stimulus; 2) a stimulus with well defined physical properties, and the method by which the stimulus is delivered; 3) instructions which guide the subject in what characteristics to attend to and what features to report, and 4) investigator/examiner who provides the instruction and controls the delivery of the stimulus.

The terms “probe” or “thermal probe”, as used herein, refer to parts of a device designed for neurological testing in subjects with peripheral neuropathies affecting sensory perception of temperature. The probe is capable of producing a range of temperatures, which may be perceived by the subjects as cold, neutral, warm, painfully hot, or anything in between. In one embodiment, the probe is capable of producing calibrated temperatures in the range of about 1 degree Celsius (1° C.) to about 60 degrees Celsius (60° C.) with an accuracy of ±1 degree Celsius (1° C.). In another embodiment, the probe is capable of producing calibrated temperatures in the range of about 30 degrees Celsius (30° C.) to about 50 degrees Celsius (50° C.) with an accuracy of ±1 degree Celsius (1° C.). The desired temperature is selected by an examiner or an investigator.

In some embodiments, the present invention contemplates that the thermal probe (i.e. temperature sensor) is incorporated into a heating block. The heating block may have a variety of shapes and/or combinations of shapes, including square, rectangular, oval, oblong, etc. The size of the heating block that comes in contact with the skin is typically between about 6 cm² and about 8 cm², with volume of the block dependent on the volumetric heat capacity of the material used, but preferably sufficient to retain the temperature within the specified accuracy for the duration of the test (e.g. 5 seconds), provided the heat loss to the skin roughly equals the energy supplied by the heating element. Optionally, the heating block may be detachable from the main device for ease of cleaning, service, or storage. Several different heating blocks, varying in size, shape, mass, volume, etc. may be optionally provided for different test conditions.

As used herein, the term “subject” encompasses mammals and non-mammals. Examples of mammals include, but are not limited to, any member of the mammalian class: humans (including patients and volunteers), non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like.

In the practice of the methods, the examiner typically applies the probe to an area of the subject's skin for a short period of time in order to assess whether the subject has any loss of temperature sensation in that area. In some embodiments, the thermal stimulus from the probe is applied for a period of between about 1 second and about 10 seconds. In other embodiments, the thermal stimulus from the probe is applied for a period of about 5 seconds. The desired temperature of the probe (i.e., the temperature of the thermal stimulus) is selected by an examiner (investigator). In some embodiments, thermal stimulus is applied using probe temperature of about 38° C. In other examples, thermal stimulus is applied using probe temperature of about 47° C. In previous studies, it has been established that the tested subjects perceived the 38° C. temperature as “warm” about 95% of the time. Also in previous studies, it has been established that the tested subjects perceived the 47° C. temperature as “painfully hot” about 95% of the time. In the practice of the methods, the perception of temperature at the stimulated site may be compared with the perception of temperature at another, non-stimulated site in the subject. The subjects provide individual-specific reports, thereby providing subject-specific assessment. This approach is important for phenotyping an individual subject with respect to the subject's sensory perception of pain.

“Feedback data point recorder” refers in its broadest sense to anything that can record sensation data that are provided by the subject. When the subject indicates particular intensity of heat sensation, the feedback data can be recorded in a variety of ways, including but not limited to analog recordings, digital recordings, audio recordings, visual recordings, mechanical recordings, electrical recordings, any combinations thereof, etc. The data can be recorded directly by the subject; alternatively, or in addition, the data can be recorded indirectly, by a recorder; the data can be recorded manually, in automated ways, etc. For example, the subject may verbally communicate the level of heat sensation during various levels of heat stimulation, and the corresponding data points may be recorded either directly by the subject or indirectly by an examiner.

In one embodiment, the device of the present invention has timer functionality (e.g., a display timer) to help the examiner in performing the test. The timer is used for display and/or measurement of time. Alternatively, or in addition, there may be one or more audio signal(s), such as a simple beep or synthesized voice, specifying the beginning and end of measurements.

In one embodiment, the power to the components of the device, including the probe, is obtained from a conventional 120 V AC outlet using a power adapter with regulated DC voltage. For example, the power adapter may output regulated DC voltage of 12 V and no less than 500 mA of current. Any other power source sufficient to power the device of a specific configuration may be used, such as batteries, other industrial voltages (such as European 220V/50 Hz), etc.

The heat in the probe is generated as a byproduct of electric current running through a resistive component. In some embodiments of the invention, a high power metal film resistor is used for this purpose. Any other device capable of producing heat, such as wire coil, Peltier element, semiconductor crystal, etc., may be used as well.

The resistor may be positioned in direct thermal contact with the metal cap, which is applied to the subject's skin. The metal cap may be made using any type of metal suitable for such diagnostic applications, including but not limited to brass, stainless steel, metal alloys, etc. In choosing components for the probe, a special consideration is made to make sure that any component that is in direct thermal contact with the brass cap (and hence with the subject's skin) is well insulated from the electrical circuit used to supply the current.

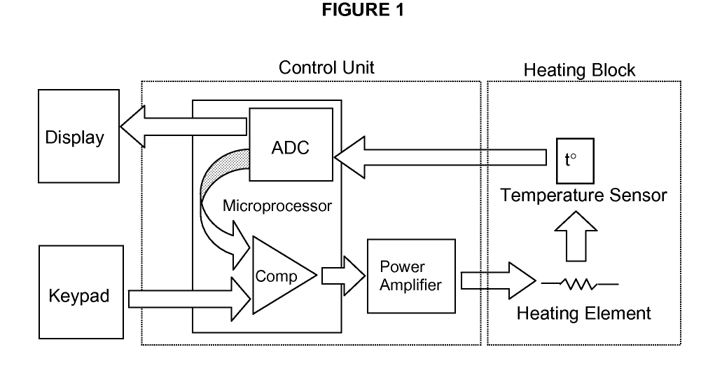

In some embodiments, the probe includes two main components at the business end of the probe; these are (i) a resistor and (ii) a temperature sensor. The design of the resistor is such that its metal casing is insulated from the resistive film by a layer of any dielectric material or combination of materials. An example of a resistor useful for practicing the invention is TCH35P5R10JE, which is made by Ohmite, Rolling Meadows, Ill. The casing of the temperature sensor is made out of plastic and is therefore insulated. Alternatively, the elements in the metal casing may be insulated from the business end of the probe by appropriate insulation, e.g. one made of plastic, ceramic compounds, etc. One embodiment of a block diagram of the device of the present invention is shown in FIG. 1.

In some embodiments, it is contemplated that the device of the present invention includes a control unit. The control unit includes two or more components. In one embodiment, the control unit is composed of a single-crystal microprocessor controller (SCMC) that includes an analog-to-digital converter (ADC), and a power amplifier. The control unit may optionally include some kind of memory component, and/or data storage unit. The temperature sensor, which is preferably an integral part of the heating block, generates voltage proportional to the actual temperature (T_{actual}) of the heating block. The temperature sensor is typically a factory-calibrated component with a specified accuracy. The ADC converts the voltage into a number equal to degrees of Celsius (° C.), thus providing a digital readout for the temperature. The temperature readout is then compared with the target temperature (i.e., set point temperature) entered by the operator (which can be entered via a keypad, a dial, up and down buttons, etc.), and the difference in the two temperatures is used to drive the power amplifier, which controls the current through the heating element. A negative feedback loop is thus effected, which provides tight control of the temperature of the heating block. The actual temperature of the heating block is then displayed in degrees of Celsius (° C.) on the display. The display may also be used to indicate the time during which the probe has been applied to the skin.

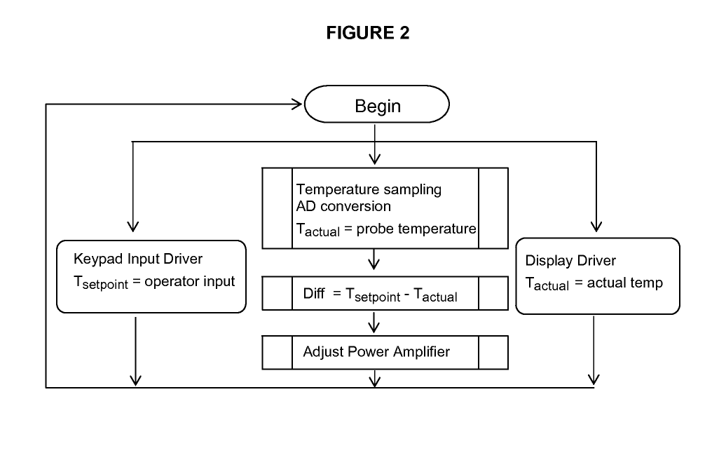

FIG. 2 is a simplified flow diagram of one example of a microcode program (MP) that runs on the single-crystal microprocessor controller. In the embodiment shown in FIG. 2, the SCMC runs three separate processes in parallel. First, the keypad driver handles the subject, i.e. the user input. Second, the display driver displays the actual temperature on the display. Third, the remaining process effects the negative feedback loop and drives the power amplifier.

In one aspect, the invention provides implementation of a failsafe mechanism for the quantitative sensory testing (QST) device. The device is based on the concept of a negative feedback loop (NFL), which ensures that any discrepancy in the temperature readout by a temperature sensor (T_{actual}) from the temperature specified by the examiner (referred to as Tₛₑₜₚₒᵢₙₜ, set point or target temperature) is eliminated by adding this discrepancy to the power amplifier input (see FIG. 2). Thus, positive discrepancy (Tₛₑₜₚₒᵢₙₜ>T_{actual}) will result in more current to the heating element, thus raising T_{actual}, and negative discrepancy (Tₛₑₜₚₒᵢₙₜ<T_{actual}) will result in less current to the heating element, thus lowering T_{actual}.

The effective and safe operation of the device is based on the integrity of the NFL, which is the same as the integrity of all blocks and connections that comprise the loop ADC->Comp->Power Amplifier->Heating element->Temperature sensor->ADC, as shown in FIG. 1. In addition, failure or proper interface (IF) with the examiner (ADC->Display, or Keypad->Comp, as shown in FIG. 1), will also result in a functional failure of the device. Failure of any of the components of the loop or the interface may result in an incorrect temperature applied to the subject's skin. It is particularly important to make sure that the subject's skin is not exposed to temperature extremes (e.g., greater than 50 degrees Celsius).

Any electronic circuit designed to implement such temperature control will be susceptible to faulty blocks and connections. Many traditional approaches to increasing the reliability of the circuitry and minimizing probability of failure exist. As representative examples, redundant circuitry and failsafe circuitry are more commonly used. The former comprises additional copies of the main control circuit ready to take over the main circuit in case of main circuit failure. The latter provides power cutoff in case of main circuit failure. In both example cases, the implementation of a safety design is more than trivial. Thus, the present device describes a novel way of implementing failsafe for a reliable NFL and IF by combining the two in one integral design.

Apart from design considerations described above, the main concern for the safety of the thermal probe is ensuring that the probe does not heat above the specified temperature, thus minimizing the risk of burn injury to the subject. The examiner will never apply the probe to the subject's skin without first making sure that the temperature readout is what is desired. The examiner will then apply the probe to his/her own skin to verify that the temperature is not extreme. While it is theoretically possible that a faulty device can produce an extreme temperature, an automatic safety mechanism is inherent to the very design of the device. The display driver process run by the SCMC (see FIG. 2) is a dynamic display driver. Only if the SCMC is operating properly will any data appear on the display. The presence of the correct temperature readout on the display thus ensures not only that the temperature of the heating block is as required, but also that the control circuitry is operating correctly, which makes production of the extreme temperature extremely unlikely.

This invention provides methods for obtaining comprehensive information about neuropathic pain in a quantitative manner. In some embodiments, the methods may include using the device to obtain data with respect to one or more of the following: 1) systematic measurement of neuropathic pain symptoms in subjects by means of specific tools; 2) systematic measurement of neuropathic pain signs in subjects by means of Quantitative Pain Sensory Testing; 3) analysis to validate neuropathic pain signs, and optionally to begin to classify subjects based on symptoms and signs.

The devices, systems, and methods of the present invention are useful for conducting diagnostic assays for quantitative sensory testing of neuropathic pain in subjects. The invention contemplates use of these devices, systems, and methods in routine patient evaluation, therapy delivery, clinical trials, etc., to quantify neuropathic pain. These may be used anywhere where QST may be used for the detection of sensory deficits, such as those that develop from small fiber disease in diabetic polyneuropathy, for detecting deficits in patients with diabetic and HIV neuropathies, as well as in clinical trials. In particular, recently established protocols and normative data for QST can be used for detection and measurement of both positive and negative sensory phenomena, both of which contribute to neuropathic pain.

The devices, systems, and methods of the present invention are useful for measuring something that there was previously no need for. In one aspect, the devices, systems, and methods of the present invention take advantage of intensity stimulus rating, as provided by the tested subjects, vs. the previously used devices and methods that are focused on threshold detection. In preferred embodiments of the invention, the devices, systems, and methods of the present invention use the tested subject as a reference. The subject may provide feedback with respect to heat sensation correlating to heat stimuli that are applied over a range of temperatures, e.g. from about 1° C. to about 60° C., or from about 30° C. to about 50° C. The provided feedback is compared to normative data, standardized or predetermined values. In contrast to the detection of just one value corresponding to the threshold of sensation (e.g. heat), the heat sensation data corresponding to a range of applied temperatures, and referenced to the subject, as provided for in the present invention, can be used, e.g., for sensory mapping of the tested subject, to acquire a pattern of sensory pain abnormalities, and to provide subject-specific reports, thus providing subject-specific assessment with respect to sensory perception of pain.

The evaluation of the patient with neuropathic pain involves establishing whether the pain is indeed neuropathic, determining the type of neuropathic pain, and identifying the characteristics of the sensory disturbance. The neuropathic pain questionnaire, physical examination, and QST are integral to this process. During initial screening for neuropathic pain it is important to answer the question whether the patient has a neuropathic pain disorder. This can be determined on the basis of symptom scales and bedside examination. At this point it is critical to identify positive and negative sensory phenomena which are consistent with neuropathic pain. For example, one can begin with simple scales such as IDPain (Portenoy, 2006, Curr. Med. Res. Opin. 22: 1555-1565), and then proceed to more extensive scales such as the NPQ, Neuropathic pain questionnaire (Krause and Backonja, 2003, Clinical Journal of Pain 19: 306-314) if the IDPain identifies features of neuropathic pain. The examiner next addresses the question of what neuropathic pain disorder the patient may have. Pain diagnosis is made on the basis of history and physical examination, using tools such as pain diagrams, neurological symptoms, and bedside examination, which employs methods of stimulation shared with QST such as light brush (LB) and punctuate/pinprick (PP) testing. Mapping the area of abnormalities is conducted for purposes of both diagnosis and quantification. The pain history, pain diagram, bedside examination, and mapping allow the examiner to identify sites for subsequent QST.

In general, the QST principles and the type of information obtained by QST include the following. As a psychophysical methodology, there are many aspects and components that have to be considered, including the basic elements: subject, stimulus, instruction, examiner, as well as tissue and organs that need to be tested, type of stimulus and method of application of the stimulus, and the type of information obtained from QST.

In general, for QST to be successfully conducted there are specific prerequisites from each of these elements, as follows. First, the subject has to be able to understand instructions about each step and the task of testing. Prior to testing the subject is trained in the procedure. During the entire testing procedure the subject has to be alert, attentive, and able to respond as instructed. It is these characteristics of a subject's participation which the examiner has to utilize to determine whether the study is valid. Sources of variability which can adversely influence a patient's responses include, but are not limited to, cognitive deficits, inattention, anxiety, and intention to deceive. Some of these elements, such as cognitive impairment, can be screened for, while others, such as deception are more difficult to identify and control for. At the end of each testing session, the examiner should record their judgment about validity of the testing based on patient's participation during the testing. Second, the physical properties of the stimulus have to be standardized, including the area of application, intensity, duration and rate of stimulus application. It is important to consider that there are many types of stimuli, including mechanical (brush, pressure, pinprick and punctate), thermal (innocuous warm and cold and noxious heat and cold pain), chemical (capsaicin, menthol, histamine), and electrical. Parameters such as type of stimulus, tissue stimulated, area of stimulation, the size and other physical prosperities of the probe, stimulus duration, interstimulus interval, and length of the testing procedure have been specifically investigated as variables during QST studies. Third, the instructions given to the subject are critical and must be standardized, simple, and unambiguous. The subject has to demonstrate during the training session that he/she understands and is able to participate in the testing. Fourth, the investigator has to be trained in each step of the QST procedure and must be able to demonstrate proficiency in conducting the test. The investigator has to be able to communicate instructions to the subject, to conduct the testing procedure, and to record data from testing. The investigator has to be attentive to the entire testing procedure and to the subject's responses.

Although many of these elements are explained in most of the published literature, widely accepted standardization of these elements for QST in routine clinical research and practice have not been adopted, impeding adoption of QST as a clinical tool. There are number of precautions that should be kept in mind while performing QST. There are a few factors which must be controlled by the examiner, such as consistency of instruction and preparation of the exam environment. There are also factors that examiner cannot control but which should be recorded, such as the patient's alertness, attention and cooperation, and changes in the course of the disease. At the end of each testing session, the examiner should record their judgment about validity of the testing based on patient's participation during the testing, similar to any testing which requires patient's involvement.

Several technical considerations are important in the interpretation of QST results. First, test-retest variability has to be interpreted in the light of the dynamic nature of pain. Second, it is important to standardize instructions. Third, QST results should be interpreted in reference to normative data, and as such indicate whether the patient has a sensory deficit (elevated threshold), or allodynia/hyperalgesia (decreased threshold) from innocuous and noxious stimuli, respectively. In conclusion, numerous factors, including the subject, stimulus, instructions, examiner, the tissue and organs that need to be tested, the type and method of application of the stimulus, and the type of information obtained from QST are important and need to be considered when performing QST.

While conducting QST, it is important to understand the type and extent of sensory abnormalities, including both positive and negative phenomena, in the area of interest. For the interpretation of QST findings one can use normative data as well as an unaffected control site on the patient, such as the contralateral limb or trunk. When performing QST, the number of test sites is usually limited by constraints of time and patient cooperation. Thus, symptom assessment and bedside sensory exam provide the foundation and context for the selection of the QST testing site. QST provides information about the type of sensory abnormalities, such as deficits and positive sensory phenomena, while the pattern of sensory pain abnormalities revealed by pain diagrams and pattern of abnormalities detected during sensory mapping of abnormalities on the exam provide additional complementary information.

With respect to the QST principles and methods, as a psychophysical methodology, there are many aspects and components that are considered, including the basic elements (subject, stimulus, instruction, examiner), tissue and organs that need to be tested, type of stimulus and method of application of the stimulus, and the type of information obtained from QST. QST has several elements: subject, stimulus, instruction, examiner and the outcome of testing. In general, for QST to be successfully conducted there are specific prerequisites from each of these elements, as explained below. First, the subject has to be able to understand instructions about each step and the task of testing. Prior to testing the subject is trained in the procedure. During the entire testing procedure the subject has to be alert, attentive, and able to respond as instructed. Second, the physical properties of the stimulus have to be standardized, including the area of application, intensity, duration and rate of stimulus application. It is important to consider that there are many types of stimuli, including mechanical (brush, pressure, pinprick and punctate), thermal (innocuous warm and cold and noxious heat and cold pain), chemical (capsaicin, menthol, histamine) and electrical. Third, the instructions given to the subject are critical and must be standardized, simple, and unambiguous. The subject has to demonstrate following the training session that they understand and are able to participate in the testing. Fourth, the investigator has to be trained in each step of the QST procedure and must be able to demonstrate proficiency in conducting the test. The investigator has to be able to communicate instructions to the subjects, to conduct the testing procedure, and to record data from testing. The investigator has to be attentive to the entire testing procedure and to patient's responses. Although these elements are explained in most of the published literature, widely accepted standards for QST in routine clinical research and practice have not been adopted.

There are a number of precautions that should be kept in mind while performing QST. Several factors are typically controlled by the examiner, such as consistency of instruction and preparation of the exam environment. There are also factors that examiner cannot control but which should be recorded, such as the patient's alertness, attention and cooperation, and changes in the course of the disease. At the end of each testing session, the examiner should preferably record their judgment about validity of the testing.

Several technical considerations are important in the interpretation of QST results. First, test/retest variability has to be interpreted in the light of the dynamic nature of pain. Second, it is important to standardize instructions. Third, QST results should be interpreted in reference to normative data, and as such indicate whether the patient has a sensory deficit (elevated threshold), or allodynia/hyperalgesia (decreased threshold) from innocuous and noxious stimuli, respectively. Either established norms or, in the case of unilateral symptoms, the patient's asymptomatic side, could be used for normative data, keeping in mind that in some conditions the asymptomatic side may not be normal. In summary, numerous factors, including the subject, stimulus, instructions, examiner, the tissue and organs that need to be tested, the type and method of application of the stimulus, and the type of information obtained from QST are important and need to be considered when performing QST. The published literature serves as the foundation for QST that can be used in clinical research and practice of neuropathic pain.

In some embodiments, the methods of bedside testing of specific sensory modalities include the use of thermal stimuli, which may be delivered using the devices and the methods of the present invention. The sensations of coldness or warmth when the skin is cooled or heated outside the thermoneutral zone (31° C.-36° C.) are due to activation of Aδ and C thermoreceptors, respectively. Thermoreceptors have free nerve endings in the epidermis. The threshold for heat pain is approximately 45° C. and the threshold for cold pain varies from less than 0° C. to more than 15° C. Both A-δ mechanoheat nociceptors (AMH type II) and C polymodal nociceptors are believed to mediate painful thermal stimuli. Not wanting to be bound by the following theory, two pain qualities to thermal stimuli have been described. The first pain is easily localized, sharp pricking pain mediated by myelinated cold-specific A delta fibers, whereas the second pain is poorly localized, burning pain mediated by unmyelinated warm-specific C-fibers.

The thermal thresholds and thermal pain thresholds vary inversely with size and duration of the stimulus. These well-recognized features are referred to as spatial and temporal summation, respectively. Because of the phenomenon of spatial sensation, it is important to maintain a constant probe size in any comparative study of thermal threshold.

### EXAMPLES

It is to be understood that this invention is not limited to the particular methodology, protocols, subjects, or reagents described, and as such may vary. It is also to be understood that the terminology used herein is for the purpose of describing particular embodiments only, and is not intended to limit the scope of the present invention, which is limited only by the claims. The following examples are offered to illustrate, but not to limit the claimed invention.

Study of Positive Sensory Phenomena in Neuropathic Pain

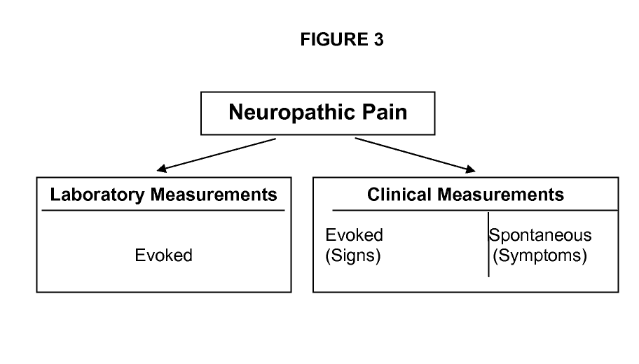

In the practice of the present invention, positive sensory signs can be validated by sensory symptoms since they have been established for neuropathic pain (FIG. 3) and validated in a number of studies (Bennett et al. 2006, Pain 127: 199-203). In this example, two types of information that are obtained in the evaluation of patients with neuropathic pain are obtained in sequential fashion. First, self-reported symptoms representing neuropathic pain that patients experience due to neurological dysfunctions and disorders are quantified by means of tools developed specifically for neuropathic pain. These tools include the Neuropathic Pain Questionnaire, Neuropathic Pain Scale, Neuropathic Pain Symptoms Inventory and PainDetect (Krause and Backonja, 2003, Clin. J. Pain 19: 306-314; Galer and Jensen, 1997, Neurology 48: 332-338; Bouhassira et al., 2004, Pain 108: 248-257; Bennett et al., 2006, Pain 127:199-203). These instruments are unique, and include a variety of assessments, though all of them assess neuropathic pain. All of these tools have been validated in the process of their development.

Second, sensory signs are the product of an examination conducted on the neuropathic pain patient by the clinician, and they are elicited or evoked. In some aspects, this invention can validate specific positive sensory evoked phenomena, such as allodynia, by applying quantitative methods in conducting the examination and anchoring it to corresponding sensory symptoms such as those described in Table 1. The main contribution of studying evoked sensory signs is that many patients are not aware of the extent of sensory disturbances, such as simultaneous existence of positive and negative phenomena in the same pain-affected area. This additional information is only revealed in a quantitative manner by applying Quantitative Pain Sensory Testing (QSPT) as described herein. QPST was specifically developed for the comprehensive measurements of the spatial extent of sensory abnormalities and the scope and severity of those abnormalities. The additional information obtained by studying evoked sensory phenomena should lead to a more complete understanding of neuropathic pain mechanisms, such as those described in Table 1. The outcome of these methods at the clinical practice level is a validation of evoked signs as a clinically useful tool, which will help to classify patients on the basis of their symptoms and signs. At the research level, such studies will further advance translational research in mechanisms of neuropathic pain by applying these methods in clinical trials. Quantitative study of specific sensory signs can lead to study of neuropathic pain mechanisms in human subjects, and until now these pain mechanisms have been studied only in the laboratory with animal models.

Thermal Probe for Testing of Warm and Heat Pain Perception

The thermal probe used in this example is designed to provide temperatures in the range of 32° C. to 50° C. In one example, only two temperatures are administered by the thermal probe, 38° C. and 47° C. These temperatures are just above the pain thresholds for up to 95% of human subjects; these temperatures are administered for up to 5 seconds or for shorter period of time if patient experiences thermal allodynia or hyperalgesia. These temperatures and length of administration are known in the art (Wallace et al., 2000, Anesthesiology 92: 75-83; Yarnitsky et al., 1995, Pain 60: 329-332; Getz Kelly et al., 2005, Muscle & Nerve 32: 179-184).

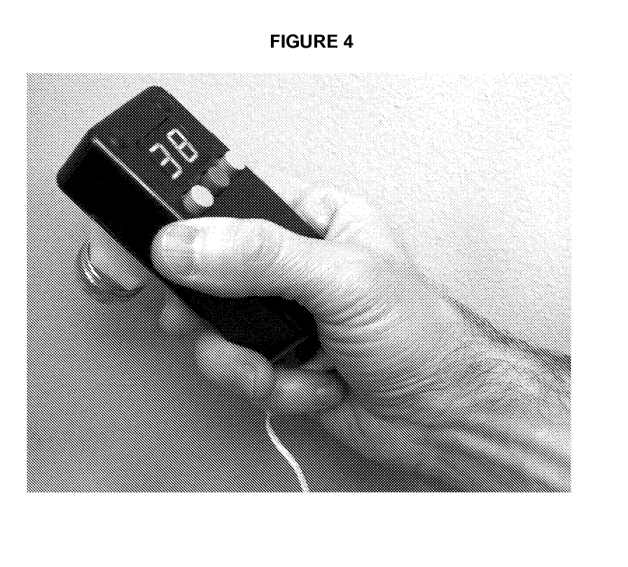

The stimuli are typically applied by trained and certified research staff. Subjects are instructed to stop the testing at any point they find it to be disagreeable. Simplicity of this testing device and its application qualifies it as a Nonsignificant Risk Device as specified by the FDA. As shown in FIG. 4, in one preferred embodiment the device of the present invention is configured for use with a human subject.

TABLE 1
Examples of symptoms and signs corresponding
to positive and negative sensory phenomena

| | Symptoms | Signs |
|---|---|---|
| Positive Sensory | Sensitive to touch | Allodynia |
| Phenomena | | |
| Negative Sensory | Numbness | Decreased sensation |
| Phenomena | | or |
| | | Loss of sensation |

QSPT can be validated in patients with neuropathic pain by applying a comprehensive quantitative approach in determining how neuropathic pain symptoms and signs contribute to the neuropathic pain experience. It is possible to conduct quantitative measurement of symptoms and signs in patients with traditional neuropathic painful disorders, including postherpetic neuralgia (PHN), painful diabetic neuropathy (PDN) and spinal cord injury (SCI) pain. One or more of these etiological diagnoses are typically selected because they are the best established human models of peripheral (PHN, PDN) and central (SCI) neuropathic pain, and by studying them it is possible to draw general conclusions about neuropathic pain. Since patients with neuropathic pain have a wide range of sensory symptoms, a battery of corresponding neuropathic pain symptom tools can be administered. In addition, comprehensive QSPT can be performed to elicit and elucidate signs.

In one exemplary design of a study using the devices and the methods of the present invention, subjects of the study are patients who experience neuropathic pain due to PHN, PDN and SCI. The subjects may be asked to complete one or more neuropathic pain symptom-specific measurement tools, including the Neuropathic Pain Questionnaire, Neuropathic Pain Scale, Neuropathic Pain Symptoms Inventory and PainDetect. The subjects are then subjected to Quantitative Pain Sensory Testing (QSPT). The primary analysis includes validation of each subject-specific neuropathic pain sensory sign with corresponding symptoms. The secondary analysis includes measurement of one or more of the following: influence of the most severe symptoms on the overall pain intensity for individual patients; influence of the most severe signs on overall pain intensity; influence of affective and functional measures on overall pain intensity; comparisons across etiologies to determine similarities and differences in symptoms and signs profiles; symptoms and signs profile for each specific patient.

In one example, patients with post-herpetic neuralgia (PHN), painful diabetic neuropathy (PDN) or pain due to spinal cord injury (SCI) are recruited at the institutional pain centers by the investigators, through advertisement in the pain centers as well as through centers such as the Office of Clinical Trials where investigators practice. Additional recruitment can be done through advertisements in the community. The subjects are clinically evaluated, and the study procedures are typically performed at the institutional research centers.

Diagnoses may be established/confirmed on the basis of standard clinical diagnostic procedures, consisting of history and physical examination, including neurological examination, and corroborated with appropriate and necessary laboratory, imaging and electrophysiological studies. Subjects included in such studies are generally medically stable and do not have significant medical or psychiatric comorbidities that would preclude them from participating in the studies.

Inclusion Criteria

Human subjects are typically 18 years of age and older, and able to provide informed consent and communicate. Pain rating inclusion criteria will typically be pain greater than 3 up to 9, as rated on the 0-10 pain scale, where 10 is the worst pain imaginable. This rating is based on the numeric pain rating scale (NPRS).

PHN: subjects with PHN will have a history of pain of at least 6 months duration in the area that was the site of a zoster rash resulting in nerve injury. In most cases, subjects experience a number of sensory abnormalities in the affected area, ranging from pain to numbness, and various degrees of hypersensitivity. Subjects with PHN must otherwise be in stable health. Pain rating inclusion criteria for PHN subjects will be pain greater than 3 up to 9, as rated on the 0-10 pain scale, where 10 is the worst pain imaginable.

PDN: subjects with diabetes mellitus and neuropathy who have a history of pain, predominantly in the lower extremities, of at least 6 months duration qualify for the diagnosis of PDN for purposes of this study. In most cases PDN is due to small fiber neuropathy, so physical examination should yield sensory abnormalities, such as pain, paresthesiae and numbness. Motor function and stretch reflex abnormalities are common but not necessary for inclusion. PDN patients in whom large fiber functions are affected, experience weakness and decreased or absent stretch reflexes, respectively, and are eligible for this study. Other causes of neuropathy will be excluded. PDN patients with pain rating of greater than 3 up to 9 on 0-10 scale will be included.

SCI pain: these are subjects with SCI and pain of at least 6 months duration. In most cases, these subjects' sensory, motor and stretch reflex abnormalities are consistent with SCI. Sensory findings range from complete loss of sensation to preservation of all sensory modalities. Motor findings range from mild weakness to complete paralysis. Stretch reflexes are most frequently increased though in a few patients they can be absent. Based on the constellation of sensory and motor findings in particular patients, diagnosis of complete versus incomplete SCI is made. It is possible to study patients with incomplete SCI who have pain at level of injury or below level of injury, or both. Subjects with SCI who have pain rating of greater than 3 up to 9 on 0-10 scale may be included in the tests.

Exclusion Criteria

One or more groups of patients may be excluded from the studies. A non-limiting list of groups of patients is described below. Patients with pain due to disorders other than PDN, PTN, or SCI, as well as unknown causes, are typically excluded. Patients with neuropathies from causes such as vasculitis, demyelinating polyneuropathies, HIV-associated neuropathy, and paraneoplastic and post-infectious neuropathies are typically excluded. Patients with chemotherapy-induced neuropathy are typically excluded. Patients who suffer from pain due to different pain mechanisms are typically excluded. Patients with other pain (at a different site) that is more severe than their PDN or PTN pain are typically excluded. Patients with a history of recent or ongoing alcohol or other drug addiction disorders (as self-reported or previously documented in the medical record) are typically excluded. Patients who are determined to have cognitive and reading impairments which would preclude them from completing questionnaires are typically excluded. Patients whose chronic medical and psychiatric comorbidities are not under optimal control, or who are currently experiencing an acute exacerbation of a medical or psychiatric comorbidity, are typically excluded.

In one example, volunteers who respond to advertisements, flyers, or via email will undergo preliminary screening for inclusion/exclusion criteria via telephone, email, and the like. For potential subjects recruited at a clinic visit, preliminary inclusion and exclusion criteria are reviewed, and the consent process begun at that time. A study visit appointment is scheduled. At the first study visit, the informed consent form is reviewed by the subject, who will be given opportunities to discuss the study with the study staff and have all questions answered. A consent form will be signed by each subject prior to any research procedures being conducted.

There may be at least two study visits. During the first visit the subject undergo a complete medical examination and will complete all questionnaires, followed by quantitative sensory pain testing, which will be performed once by each of 3 examiners. There will be a 10-15 minute break between testing by each examiner. During the second visit (approximately 2 weeks later), the subject will again complete the set of questionnaires and will undergo quantitative sensory pain testing by only one examiner. During the two weeks between visits, subjects will be asked to record up to 5 of their most severe and disturbing symptoms daily using a 0-10 scale, as many times per day as they find it relevant (but a minimum of two ratings per day), via interactive voice response system (IVRS).

In one example of the assessments of Quantitative Sensory Pain Testing (QSPT), each subject will undergo QSPT of the area affected by neuropathic pain. QSPT determines the extent of sensory abnormalities to brush, punctate stimulus, vibration, innocuous and noxious cold and heat; a map of the affected area will be outlined; neurosensory testing is performed halfway between the borders of the hyperalgesic area. One or more of the following neurosensory modalities may be performed in the following order: (i) light brush, (ii) punctate, (iii) vibration (iv) innocuous warm, (v) innocuous cool (vi) pressure algometry, (vii) noxious cold and (viii) noxious hot. This order is chosen because it tests from the least noxious stimulus—touch—to the most noxious stimulus—hot pain. Warm and hot pain may be tested using the devices of the present invention, which may be specifically designed to deliver temperatures of 38° C. and 47° C. for 5 seconds, which in the majority of subjects will produce sensations of warm and hot pain, respectively. Cool sensations are tested with the metal head of a tuning fork, which in general provides a stimulus of room temperature (approximately 24° C.). Cold pain is tested after the tuning fork has been immersed in ice cold water, to provide a stimulus of approximately 0-5° C. In a majority of subjects these stimuli produce sensations of cool and cold pain respectively. The subject will be instructed to provide a description of the stimulus felt and if it is perceived as painful. If so, the intensity of that pain will be measured, using a 0-10 scale, where 10 is the worst pain imaginable. This order will only be changed if the subject reports a particular sensory modality is the most painful to them. For example, if a subject reports that light brushing is the most painful it will be moved to the last modality tested. The reason for this change in order is to hold to the principle of testing the most noxious stimulus last. This method is a modification of the widely used method of Dixon in human psychophysical testing (Wallace et al. 1997, Anesthesiology 86: 1262-1272). This part of the evaluation should take approximately 20 to 25 minutes.

In addition to the QST, the assessments may include measurement of spontaneous symptoms. These are spontaneous subjective sensations related to pain (symptoms) will be assessed with the previously-mentioned self-administered standardized scales and questionnaires. Collectively, these scales assess general pain symptoms as well as more specific symptoms associated with neuropathic pain. Frequently associated psychological correlates of pain, including depression and anxiety, as well as impact of neuropathic pain on functioning and quality of life, may also be evaluated with standard scales and questionnaires. These include, e.g., Neuropathic Pain Symptom-specific measurement tools: Neuropathic Pain Questionnaire, Neuropathic Pain Scale, Neuropathic Pain Symptoms Inventory, PainDetect and the mechanical Visual Analogue Scale (VAS). All of these tools measure intensity of neuropathic pain symptoms and they contain similar but not identical symptom items. Other measures that may be used in the practice of the present invention are the measures of psychological well-being, quality of life and functional abilities. These include: Pain Anxiety Symptoms Scale (PASS), used to assess anxiety associated specifically with pain; Linear Analogue Self Assessment (LASA), used to assess quality of life; Pain Catastrophizing Scale (PCS), used to assess presence and severity of catastrophization as one the mechanisms patients use to deal with pain; Brief Pain Inventory short form (BPI-sf) includes detailed information on pain intensity (worst pain, least pain, average pain, and pain right now), used to assess interference of pain with patients' daily functions and quality of life. The BPI has been used in a number of pharmacological and other pain outcome studies; Center for Epidemiological Studies Depression Scale (CESDS), and may be used to assess aspects of pain related to depression. The completion of all questionnaires should take approximately 20 to 25 minutes.

The main difference from a standard clinical examination and the methods of the present invention is that the stimuli described herein are administered in a standardized manner with specifically-calibrated devices, to be administered in such a way that should evoke minimal exacerbation of pain. In addition, the tested subjects receive very specific instruction that they can stop the testing at any time. The information about pain that leads to termination of testing is one of the variables that is recorded and analyzed.

Statistical Methods and Data Analysis

Descriptive analyses of NP sensory symptoms and signs may be conducted. It is possible to use a variety of multivariate techniques, such as cluster analysis and factor analysis to identify groups in the signs and symptoms matrix. On the basis of previous published validation studies with neuropathic pain symptoms and signs, a sample size of 180 patients is sufficient and represents economically and practically feasible number to conduct such studies. The primary and secondary analyses may be conducted by assessing the association between signs and the corresponding symptoms. Regression analyses may be used to address the questions of influence of sensory and emotional items on overall pain intensity rating.

Validation Studies

Examples of validation studies of the devices and methods of the present invention are also provided in Table 2 (one tested subject) and in Table 3 (another tested subject). These examples provide samples of information obtained by the device and the QST method of the present invention during the test-retest study. It should be noted that a number of types of stimuli are used and among them are warm and heat pain stimuli applied in these examples to patients with PHN. In case of these patients predominant findings are those of sensory deficits as indicated by minus sign preceding the actual number.

TABLE 2
QST Example 1

| | | | | | Day 2 Date: 08 | |
|---|---|---|---|---|---|---|
| | Day 1 Date: 28 Dec 2007 | | | | Jan 2008 | |
| | test 1: | | test 2: | | test 3: | |
| QST | TBM | control 1 | MW | control 2 | TBM | control 3 |
| Time | ND | | ND | | ND | |
| Modality | | | | | | |
| 1. Site of testing | | | | | | |
| 2. Pain description (if none, select | cramping | | cramping | | cramping | |
| anther site) | | | | | | |
| 3. Pain intensity rating right now | 47 | | 57 | | 52 | |
| (mm VAS) | | | | | | |
| 4. Temperature of the site | 33.3 | 31.1 | 31.8 | 30.9 | 30.2 | 31.3 |
| 5. Is light brush sensation normal, | no | Yes | no | Yes | no | Yes |
| not painful | | | | | | |
| 6. Light brush testing | more | | more | | different | |
| ($&¢ for negative and mVAS for | 31 | | 64 | | 0 | |
| positive) | | | | | | |
| 7. Light brush area of allodynia (if it | NA | | NA | | NA | |
| exists) (cm2) | | | | | | |
| 8. Light brush area of deficit (if it | NA | | NA | | NA | |
| exists) (cm2) | | | | | | |
| 9. Light brush testing - summation | absent | | absent | | absent | |
| 10. Light brush testing - after- | absent | | absent | | absent | |
| sensation | | | | | | |
| 11. Vibration (score) | 56 | | 73 | | 73 | |
| 12. Vibration | less | | less | | more | |
| ($&¢ for negative and mVAS | −100 | | −100 | | 62 | |
| for positive) | | | | | | |
| 13. Cool | less | | less | | less | |
| ($&¢ for negative and mVAS for | −100 | | −100 | | −50 | |
| positive) | | | | | | |
| 14. Warm | more | | less | | less | |
| ($&¢ for negative and mVAS | −50 | | −100 | | −100 | |
| for positive) | | | | | | |
| 15. Pinprick: | more | | more | | more | |
| ($&¢ for negative and mVAS for | 72 | | 89 | | 80 | |
| positive) | | | | | | |
| 16. Pinprick testing - summation | absent | | present | | present | |
| 17. Pinprick testing - after-sensation | present | | absent | | absent | |
| 18. Pinprick area of hyperalgesia (if | NA | | NA | | NA | |
| present) (cm2) | | | | | | |
| 19. Pinprick area of deficit (if | NA | | NA | | NA | |
| present) (cm2) | | | | | | |
| 20. Cold pain | more | | more | | less | |
| ($&¢ for negative and mVAS for | 39 | | 52 | | −100 | |
| positive) | | | | | | |
| 21. Cold pain - after-sensation: | absent | | absent | | absent | |
| absent, present | | | | | | |
| 22. Heat pain | less | | less | | less | |
| ($&¢ for negative and mVAS for | −50 | | −100 | | −80 | |
| positive) | | | | | | |
| 23. Heat pain - after-sensation: | absent | | absent | | absent | |
| absent, present | | | | | | |
| 24. Pressure pain | NA | | NA | | NA | |
| ($&¢ for negative and mVAS for | NA | | NA | | NA | |
| positive) | | | | | | |
| 25. Pain description | cramping | | cramping | | cramping | |
| 26. Pain intensity rating right now | 67 | | 52 | | 76 | |
| 27. Temperature of the site | 33.2 | | 34.6 | | 29.9 | |

TABLE 3
QST Example 2

| | | | | | Day 2 Date: | |
|---|---|---|---|---|---|---|
| | Day 1 Date: Dec. 28, 2007 | | | | Jan. 07, 2008 | |
| QST | test 1: PH | control 1 | test 2: GI | control 2 | test 3: GI | control 3 |
| Time | 11:30 | 11:30 | 12:15 | 12:15 | 16:30 | 16:30 |
| Modality | | | | | | |
| 1. Site of testing | Left | Right | Left | Right | Left | Right |
| | lower | lower | lower | lower | lower | lower |
| | abdomen | abdomen | abdomen | abdomen | abdomen | abdomen |
| 2. Pain description (if | itchy | None | itchy | None | itchy | None |
| none, select anther | achy | | aching | | achy | |
| site) | | | | | | |
| 3. Pain intensity rating | 8 | | 10 | | 15 | |
| right now (mm VAS) | | | | | | |
| 4. Temperature of the | 36.8 | 37.1 | 36.2 | 35.9 | 36.2 | 36.1 |
| site | | | | | | |
| 5. Is light brush | | Yes | | Yes | | Yes |
| sensation normal, not | | | | | | |
| painful | | | | | | |
| 6. Light brush testing | Different | | Less, | | Less | |
| | | | Different | | | |
| ($&¢ for negative and | 0 | | −85 | | −80 | |
| mVAS for positive) | | | | | | |
| 7. Light brush area of | n/a | | n/a | | n/a | |
| allodynia (if it exists) | | | | | | |
| (cm2) | | | | | | |
| 8. Light brush area of | n/a | | n/a | | n/a | |
| deficit (if it exists) | | | | | | |
| (cm2) | | | | | | |
| 9. Light brush testing - | Absent | | Absent | | Absent | |
| summation | | | | | | |
| 10. Light brush | Absent | | Absent | | Absent | |
| testing - after- | | | | | | |
| sensation | | | | | | |
| 11. Vibration (score) | 1 | n/a | 2.6 | n/a | 4 | |
| 12. Vibration | Less, | Yes | Less | Yes | Less | No |
| | Different | | | | | |
| ($&¢ for negative and | −80 | | −80 | | −75 | |
| mVAS for positive) | | | | | | |
| 13. Cool | Less, | Yes | Less | Yes | Less | Yes |
| | Different | | | | | |
| ($&¢ for negative and | −90 | | −75 | | −90 | |
| mVAS for positive) | | | | | | |
| 14. Warm | Less | Yes | Less | n/a | Less | Yes |
| ($&¢ for negative and | −80 | | −80 | | −80 | |
| mVAS for positive) | | | | | | |
| 15. Pinprick: | Less, | Yes | Less | Yes | Less | Yes |
| | Different | | | | | |
| ($&¢ for negative and | −75 | | −60 | | −75 | |
| mVAS for positive) | | | | | | |
| 16. Pinprick testing - | Absent | | Absent | | Absent | |
| summation | | | | | | |
| 17. Pinprick testing - | Absent | | Present | | Absent | |
| after-sensation | | | | | | |
| 18. Pinprick area of | n/a | | n/a | | n/a | |
| hyperalgesia (if | | | | | | |
| present) (cm2) | | | | | | |
| 19. Pinprick area of | n/a | | n/a | | n/a | |
| deficit (if present) | | | | | | |
| (cm2) | | | | | | |
| 20. Cold pain | Less | No | Less | No | Less | No |
| ($&¢ for negative and | −90 | | −80 | | −75 | |
| mVAS for positive) | | | | | | |
| 21. Cold pain - after- | Absent | | Absent | | Absent | |
| sensation: absent, | | | | | | |
| present | | | | | | |
| 22. Heat pain | Less | No | Less | No | Less | No |
| ($&¢ for negative | −80 | | −80 | | −70 | |
| and mVAS for | | | | | | |
| positive) | | | | | | |
| 23. Heat pain - after- | Absent | | Absent | | Absent | |
| sensation: absent, | | | | | | |
| present | | | | | | |
| 24. Pressure pain | Yes | Yes | Yes | Yes | | n/a |
| (lbf) | 2.7 | 2.15 | 2.3 | 0 | 3 | 2.9 |
| pressure pain nailbed | | 6.4 | | 5.8 | | 9.6 |
| 25. Pain description | annoyed | | itchy, | | itchy, | |
| | | | achy | | achy | |
| 26. Pain intensity | 18 | | 15 | | 13 | |
| rating right now | | | | | | |
| 27. Temperature of | 35.5 | | 35.9 | | 30.3 | |
| the site | | | | | | |

Table 4 summarizes major modalities, receptors, and testing methods. Postulated primary mechanisms of pathological pain are listed. Many are not yet fully accepted.

TABLE 4
Summary of major modalities, receptors, and testing methods

| Sensory | | | Postulated mechanism | Testing |
|---|---|---|---|---|
| Modality | Principal receptors | Axon type | of allodynia/hyperalgesia | instruments |
| Dynamic | Meissner's | Aβ, some C | Central sensitization | Brush |
| mechanical | Pacinian | Aβ | | Cotton wisp |
| | Hair Follicle | Aβ | | Cotton swab |
| Static | Merkel | Aβ | Central sensitization | von Frey hair |
| mechanical | Ruffini | Aβ | Peripheral sensitization | |
| Puncture | Unencapsulated | Aδ | Central sensitization | Pin |
| (sharp) | | | Peripheral sensitization | |
| Pressure | Merkel (cutaneous) | Aβ | Central sensitization | Pressure |
| | Intramuscular | | | algometer |
| | afferents (deep) | | | |
| Vibration | Pacinian | Aβ | Central sensitization | Tuning fork |
| Innocuous | Unencapsulated | C | Peripheral sensitization | Heated surface |
| warm | | | | |
| Innocuous | Unencapsulated | Aδ | Peripheral sensitization | Metallic surface |
| cool | | | | at room |
| | | | | temperature |
| Noxious heat | Unencapsulated | C | Peripheral sensitization | Heated surface |
| | | A | | |
| Noxiuos cold | Unencapsulated | Cδ | Peripheral sensitization | Cooled surface |
| | | | Central sensitization | Metallic surface |
| | | | Reduced sensitization | in ice water |

Table 5 illustrates one example of a protocol for neuropathic pain sensory testing.

TABLE 5
Proposed protocol for neuropathic pain sensory testing

General steps and guiding points:

Administer a validated neuropathic pain symptoms tool, such as
neuropathic pain questionnaire or scale, prior to performing QST
Record skin temperature and physical findings (e.g., trophic changes,
color or vascular changes) at test and control sites
Record overall pain rating at beginning and end of test
Perform testing in a quiet, comfortable setting free of distractions
Instruct subject with a practice examination of a single modality in a
clinically unaffected area at start of test
Use written instructions to assure consistency
Score sensory deficits on a −100 to 0 scale and positive phenomena on a
0 +100 scale
Test from least noxious to most noxious modality
Record any abnormal or paradoxical evoked sensations
Record subject's alertness, attention, cooperation, and relevant
behavioral observations

Choose testing site based upon history and pain diagram
Choose a control site based upon a standard algorithm; e.g.,

Homologous contralateral site if pain is unilateral
Ipsilateral unaffected site if pain is bilateral

Evaluate the following, in order, using control site for comparative ratings
of perceived stimulus intensity:

Light brush in area of greatest pain

hyperesthesia, hyperesthesia, or allodynia to single stimulus
summation, after-sensation after repeated stimuli

map area of abnormal light brush sensation

record map on transparency or body diagram

vibration in area of greatest pain

hypesthesia, hyperesthesia, or allodynia to single stimulus
summation, after-sensation after repeated stimuli

cool stimulus (e.g., steel surface at room temperature) in area of greatest
pain

hypesthesia, hyperesthesia, or allodynia to single stimulus
summation, after-sensation after repeated stimuli

warm stimulus (e.g., thermo de at non-noxious temperature) in area of
greatest pain

hypesthesia, hyperesthesia, or allodynia to single stimulus
summation, after-sensation after repeated stimuli

pin in area of greatest pain

hypesthesia, hyperesthesia, or allodynia to single stimulus
summation, after-sensation after repeated stimuli

map area of abnormal pin sensation

record map on transparency or body diagram

cold pain stimulus (e.g., steel surface cooled in ice water)

hypesthesia, hyperesthesia, or hyperalgesia to single
stimulus

heat pain stimulus (e.g., thermode at noxious but non-damaging
temperature)

hypesthesia, hyperesthesia, or hyperalgesia to single stimulus

pressure pain stimulus at area of greatest pain
consider validated psychophysical tests of pain threshold at standard sites,
e.g., pressure point threshold at thumb nailbed

TABLE 6
Abbreviations

| Abbreviation | Means |
|---|---|
| ADC | Analog-to-digital converter |
| BPI-sf | Brief pain inventory short form |
| CESDS | Center for epidemiological studies depression scale |
| CRPS | Complex regional pain syndrome |
| FDA | Food and drug administration |
| IF | Interface |
| IVRS | Interactive voice response system |
| LASA | Linear analogue self assessment |
| LB | Light brush |
| MP | Microcode program |
| NFL | Negative feedback loop |
| NPQ | Neuropathic pain questionnaire |
| NPRS | Numeric pain rating scale |
| PASS | Pain anxiety symptoms scale |
| PCS | Pain catastrophizing scale |
| PDN | Painful diabetic neuropathy |
| PHN | Postherpetic neuralgia |
| PP | Punctuate/pinprick |
| QPST | Quantitative pain sensory testing |
| QST | Quantitative sensory testing |
| SCI | Spinal cord injury |
| SCMC | Single-crystal microprocessor controller |
| VAS | Visual analogue scale |

It is to be understood that this invention is not limited to the particular devices, methodology, protocols, subjects, or reagents described, and as such may vary. It is also to be understood that the terminology used herein is for the purpose of describing particular embodiments only, and is not intended to limit the scope of the present invention, which is limited only by the claims. Other suitable modifications and adaptations of a variety of conditions and parameters, obvious to those skilled in the art of neurology, neuropathy, biomedical devices, and biomedical engineering, are within the scope of this invention. All publications, patents, and patent applications cited herein are incorporated by reference in their entirety for all purposes.

What is claimed is:

1. A neuropathy diagnostic system configured for use with a human subject, the system comprising:
a probe comprising a heating element;
a control unit coupled with the probe;
an input device operatively coupled with the control unit to enable input of a target temperature to the control unit; and
a display coupled with the control unit to display the temperature of the probe;
wherein the control unit controls the energy provided to the probe to thereby change the temperature of the probe from an initial temperature to the target temperature; and
a feedback data point recorder to record the subject's indication of the intensity of heat sensation and the temperature of the probe at that point in time.

2. The system of claim 1, wherein the control unit comprises a comparator with which to calculate the difference between the target temperature and the effective probe temperature.

3. The system of claim 1, wherein the control unit calculates a difference between the target temperature and the effective probe temperature, and with that difference adjusts the effective probe temperature to substantially correspond to the target temperature.

4. The system of claim 1, wherein the control unit comprises a single-crystal microprocessor controller.

5. The system of claim 1, wherein the control unit comprises a thermocouple to measure the temperature.

6. The system of claim 1, wherein the heating element comprises a high power metal film resistor capable of heating the probe to temperatures between about 30° C. and about 50° C. for a period of between about 1 second and about 10 seconds.

7. The system of claim 1, wherein the display displays a time period during which the probe has been applied to skin of the subject.

8. The system of claim 1, wherein the control unit comprises a safety mechanism coupled with an analog-to-digital converter, wherein if the heating element malfunctions, the safety mechanism causes the display to stop displaying the effective probe temperature.

9. The system of claim 1, wherein the probe comprises a brass cap configured to safely contact the skin of the subject.

10. A method for diagnosing neuropathy in a subject, the method comprising:
contacting the subject with a probe configured to apply heat at a range of temperatures, the probe having an initial temperature;
setting a target temperature to which the probe temperature is to be changed;
providing a readout of the temperature of the probe as it is changed to the target temperature;
recording a plurality of feedback data points as the temperature of the probe is changed from the initial temperature to the target temperature, each feedback data point comprising the subject's indication of the intensity of heat sensation and the temperature of the probe at that point in time; and
comparing the plurality of feedback data points to predetermined values, to thereby detect abnormal heat sensations, if any, by the subject.

11. The method of claim 10, wherein the plurality of the data points are gathered over a range of temperature points of between about 30° C. and about 50° C.

12. The method of claim 10, further comprising the step of contacting the thermal probe to at least another point of the subject's skin to verify correlation with a neuropathic condition.

13. The method of claim 10, wherein an area of between about 1 cm² and about 10 cm² of the subject's skin is heated using the thermal probe with a target temperature point of between about 30° C. and about 50° C.

14. The method of claim 10, wherein the target temperature is applied for a period of between about 1 second and about 10 seconds.

15. The method of claim 10, further comprising the step of observing a timer of the display that displays a period of time during which the thermal probe has contacted the subject's skin.

16. The method of claim 10, further comprising the step of removing the thermal probe from contact with the subject's skin after a set period of time.

17. The method of claim 10, further comprising the steps of contacting different points on the subject's skin, to provide a sensory map.
